(19) United States
(12) Patent Application Publication
(10) Pub. No.: US 2024/0337051 A1
(43) Pub. Date: Oct. 10, 2024

(54) NONWOVEN FABRICS INCLUDING RECYCLED POLYPROPYLENE

(71) Applicant: Berry Global, Inc., Evansville, IN (US)

(72) Inventors: Ralph A. Moody, III, Mooresville, NC (US); Mehmet Selcuk Sinangil, Mooresville, NC (US); James Ernest Sparks, Buena Vista, VA (US); Michael Wade Hayes, Buena Vista, VA (US); Christopher Bradley Walter, Stuarts Draft, VA (US); Sergio Rafael Diaz de Leon Izquierdo, Clayton, NC (US)

(21) Appl. No.: 18/627,732

(22) Filed: Apr. 5, 2024

Related U.S. Application Data
(60) Provisional application No. 63/457,522, filed on Apr. 6, 2023.

(51) Int. Cl. D04H 1/4291; A61F 13/15; A61F 13/494; A61L 15/26; A61L 15/42; D01F 1/10; D01F 6/06; D04H 1/4374; D04H 1/4382; D04H 1/56; D04H 3/16

(52) U.S. Cl. CPC D04H 1/4291; A61F 13/49413; A61L 15/26; A61L 15/42; D01F 1/10; D01F 6/06; D04H 1/4374; D04H 1/43838; D04H 1/56; D04H 3/16; A61F 2013/15292; A61F 2013/15959; A61F 2013/15967; D10B 2321/022; D10B 2401/063; D10B 2509/026

(57) ABSTRACT

Nonwoven fabrics are provided that include a first nonwoven layer comprising a first plurality of fine fibers. The first plurality of fine fibers comprises a first polymeric material including (i) a first polymer component and (ii) optionally one or more first additives, wherein the first polymer component comprises a first recycled-polypropylene (rPP) comprising a visbroken spunbond grade polypropylene having a first melt flow rate (MFR) before visbreaking and second MFR after visbreaking, and wherein the second MFR is larger than the first MFR.

### TECHNICAL FIELD

Embodiments of the presently-disclosed invention relate generally nonwoven fabrics including at least one layer comprising a plurality of fine fibers, such as meltblown and/or melt-fibrillated fibers, comprising recycled-polypropylene (rPP) comprising a visbroken spunbond grade polypropylene.

### BACKGROUND

Nonwoven fabrics are used in a wide range of applications including, inter alia, personal hygiene products, cleaning wipes, filtration media, and soil stabilization fabrics. Many of these fabrics are formed completely or predominantly from virgin fossil fuel based polymers, such as polypropylene. In this regard, there has been increased interest in using recycled plastics in a variety of disposable products. Plastic recycling generally includes recovering scrap or waste plastic and reprocessing the material into useful products. Since the majority of plastic is non-biodegradable, recycling is part of global effort to reduce plastic waste that will either be burned, placed in a landfill, or find its way into one of the world's oceans. In some instances, the use of recycled plastics may be undesirably associated with products that exhibit reduced physical properties in comparison to products formed entirely from virgin plastics.

Accordingly, there remains a need in the art for nonwoven fabrics that may comprise a large amount of recycled plastic (e.g., recycled polyolefin) without sacrificing the desired physical properties required for the particular end-use application.

### SUMMARY OF INVENTION

One or more embodiments of the invention may address one or more of the aforementioned problems. Certain embodiments according to the invention provide a nonwoven fabric comprising a first nonwoven layer comprising a first plurality of fine fibers, in which the first plurality of fine fibers comprise a first polymeric material including (i) a first polymer component and (ii) optionally one or more first additives. The first polymer component comprises a first recycled-polypropylene (rPP) comprising a visbroken spunbond grade polypropylene having a first melt flow rate (MFR) before visbreaking and second MFR after visbreaking, and wherein the second MFR is larger than the first MFR.

In another aspect, certain embodiments according to the invention provide a method of making a nonwoven fabric, such as those described and disclosed herein, comprising the following: (a) forming a first polymeric melt comprising (i) a first polymer component, wherein the first polymer component comprises a first spunbond grade recycled-polypropylene (rPP) having a first melt flow rate (MFR); and (ii) one or more first additives, wherein the one or more first additives comprises a first viscosity reducing agent; (b) forming a first vis-broken polymeric melt comprising a step of subjecting the first spunbond grade rPP to a visbreaking operation via extruding the first polymer melt at an elevated temperature, the first spunbond grade rPP has a second MFR after the visbreaking operation, and wherein the second MFR is larger than the first MFR; (c) producing a first nonwoven layer comprising a first plurality of fine fibers, such as via a meltblowing operation and/or a melt-film fibrillation operation; and (d) consolidating the plurality of plurality of fine fibers to form the nonwoven fabric, such as those described and disclosed herein.

In yet another aspect, certain embodiments of the invention provide an article comprising a personal hygiene article comprising a nonwoven fabric such as those described and disclosed herein. The personal hygiene article may comprises a diaper or pull-up. The personal hygiene article may include a barrier leg cuff (BLC), in which the BLC is formed at least in part by the nonwoven fabric, such as those described and disclosed herein.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates a spunbond-meltblown-spunbond nonwoven fabric in accordance with certain embodiments of the invention.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The terms “substantial” or “substantially” may encompass the whole amount as specified, according to certain embodiments of the invention, or largely but not the whole amount specified (e.g., 95%, 96%, 97%, 98%, or 99% of the whole amount specified) according to other embodiments of the invention.

The terms “polymer” or “polymeric”, as used interchangeably herein, may comprise homopolymers, copolymers, such as, for example, block, graft, random, and alternating copolymers, terpolymers, etc., and blends and modifications thereof. Furthermore, unless otherwise specifically limited, the term “polymer” or “polymeric” shall include all possible structural isomers; stereoisomers including, without limitation, geometric isomers, optical isomers or enantionmers; and/or any chiral molecular configuration of such polymer or polymeric material. These configurations include, but are not limited to, isotactic, syndiotactic, and atactic configurations of such polymer or polymeric material. The term “polymer” or “polymeric” shall also include polymers made from various catalyst systems including, without limitation, the Ziegler-Natta catalyst system and the metallocene/single-site catalyst system. The term “polymer” or “polymeric” shall also include, in according to certain embodiments of the invention, polymers produced by fermentation process or biosourced.

The terms “elastomer” or “elastomeric”, as used interchangeably herein, may comprise any material that upon application of a biasing force, can stretch to an elongated length of at least 110% or even to 125% of its relaxed, original length (i.e., can stretch to at least 10% or even 25% more than its original length), without rupture or breakage. Upon release of the applied force, for example, the material may recover at least 40%, at least 60%, or even at least 80% of its elongation. In certain embodiments of the invention, the material may recover from about 20% to about 100% of its elongation, from about 25% to about 95% of its elongation, from about 30% to about 90% of its elongation, from about 40% to about 80% of its elongation, or from about 50% to about 70% of its elongation. For example, a material that has an initial length of 100 mm can extend at least to 110 mm, and upon removal of the force would retract to a length of 106 mm (e.g., exhibiting a 40% recovery). Example elastomers may include Vistamaxx™ propylene-based elastomers (commercially available form ExxonMobile), which comprise copolymers of propylene and ethylene. Vistamaxx™ propylene-based elastomers, for example, comprise isotactic polypropylene microcrystalline regions and random amorphous regions.

The terms “nonwoven” and “nonwoven web”, as used herein, may comprise a web having a structure of individual fibers, filaments, and/or threads that are interlaid but not in an identifiable repeating manner as in a knitted or woven fabric. Nonwoven fabrics or webs, according to certain embodiments of the invention, may be formed by any process conventionally known in the art such as, for example, meltblowing processes, spunbonding processes, needle-punching, hydroentangling, air-laid, and bonded carded web processes. A “nonwoven web”, as used herein, may comprise a plurality of individual fibers that have not been subjected to a consolidating process.

The terms “fabric” and “nonwoven fabric”, as used herein, may comprise a web of fibers in which a plurality of the fibers are mechanically entangled or interconnected, fused together, and/or chemically bonded together. For example, a nonwoven web of individually laid fibers may be subjected to a bonding or consolidation process to bond at least a portion of the individually fibers together to form a coherent (e.g., united) web of interconnected fibers.

The term “consolidated” and “consolidation”, as used herein, may comprise the bringing together of at least a portion of the fibers of a nonwoven web into closer proximity or attachment there-between (e.g., thermally fused together, chemically bonded together, and/or mechanically entangled together) to form a bonding site, or bonding sites, which function to increase the resistance to external forces (e.g., abrasion and tensile forces), as compared to the unconsolidated web. The bonding site or bonding sites, for example, may comprise a discrete or localized region of the web material that has been softened or melted and optionally subsequently or simultaneously compressed to form a discrete or localized deformation in the web material. Furthermore, the term “consolidated” may comprise an entire nonwoven web that has been processed such that at least a portion of the fibers are brought into closer proximity or attachment there-between (e.g., thermally fused together, chemically bonded together, and/or mechanically entangled together), such as by thermal bonding or mechanical entanglement (e.g., hydroentanglement) as merely a few examples. Such a web may be considered a “consolidated nonwoven”, “nonwoven fabric” or simply as a “fabric” according to certain embodiments of the invention.

The term “staple fiber”, as used herein, may comprise a cut fiber from a filament. In accordance with certain embodiments, any type of filament material may be used to form staple fibers. For example, staple fibers may be formed from polymeric fibers, and/or elastomeric fibers. Non-limiting examples of materials may comprise polyolefins (e.g., a polypropylene or polypropylene-containing copolymer), polyethylene terephthalate, and polyamides. The average length of staple fibers may comprise, by way of example only, from about 2 centimeter to about 15 centimeter.

The term “spunbond”, as used herein, may comprise fibers which are formed by extruding molten thermoplastic material as filaments from a plurality of fine, usually circular, capillaries of a spinneret with the diameter of the extruded filaments then being rapidly reduced. According to an embodiment of the invention, spunbond fibers are generally not tacky when they are deposited onto a collecting surface and may be generally continuous as disclosed and described herein. It is noted that the spunbond used in certain composites of the invention may include a nonwoven described in the literature as SPINLACE®. Spunbond fibers, for example, may comprises continuous fibers.

As used herein, the term “continuous fibers” refers to fibers which are not cut from their original length prior to being formed into a nonwoven web or nonwoven fabric. Continuous fibers may have average lengths ranging from greater than about 15 centimeters to more than one meter, and up to the length of the web or fabric being formed. For example, a continuous fiber, as used herein, may comprise a fiber in which the length of the fiber is at least 1,000 times larger than the average diameter of the fiber, such as the length of the fiber being at least about 5,000, 10,000, 50,000, or 100,000 times larger than the average diameter of the fiber.

The term “meltblown”, as used herein, may comprise fibers formed by extruding a molten thermoplastic material through a plurality of fine die capillaries as molten threads or filaments into converging high velocity, usually hot, gas (e.g. air) streams which attenuate the filaments of molten thermoplastic material to reduce their diameter, which may be to microfiber diameter, according to certain embodiments of the invention. According to an embodiment of the invention, the die capillaries may be circular. Thereafter, the meltblown fibers are carried by the high velocity gas stream and are deposited on a collecting surface to form a web of randomly disbursed meltblown fibers. Meltblown fibers may comprise microfibers which may be continuous or discontinuous and are generally tacky when deposited onto a collecting surface. Meltblown fibers, however, are shorter in length than those of spunbond fibers.

The term “melt fibrillation”, as used herein, may comprise a general class of making fibers defined in that one or more polymers are molten and may be extruded into many possible configurations (e.g. co-extrusion, homogeneous or bicomponent films or filaments) and then fibrillated or fiberized into a plurality of individual filaments for the formation of melt-fibrillated fibers. Non limiting examples of melt-fibrillation methods may include melt blowing, melt fiber bursting, and melt film fibrillation. The term “melt-film fibrillation”, as used herein, may comprise a method in which a melt film is produced from a melt and then a fluid is used to form fibers (e.g., melt-film fibrillated fibers) from the melt film. Examples include U.S. Pat. Nos. 6,315,806, 5,183,670, 4,536,361, 6,382,526, 6,520,425, and 6,695,992, in which the contents of each are incorporated by reference herein to the extent that such disclosures are consistent with the present disclosure. Additional examples include U.S. Pat. Nos. 7,628,941, 7,722,347, 7,666,343, 7,931,457, 8,512,626, and 8,962,501, which describe the Arium™ melt-film fibrillation process for producing melt-film fibrillated fibers (e.g., having sub-micron fibers).

The term “layer”, as used herein, may comprise a generally recognizable combination of similar material types and/or functions existing in the X-Y plane.

All whole number end points disclosed herein that can create a smaller range within a given range disclosed herein are within the scope of certain embodiments of the invention. By way of example, a disclosure of from about 10 to about 15 includes the disclosure of intermediate ranges, for example, of: from about 10 to about 11; from about 10 to about 12; from about 13 to about 15; from about 14 to about 15; etc. Moreover, all single decimal (e.g., numbers reported to the nearest tenth) end points that can create a smaller range within a given range disclosed herein are within the scope of certain embodiments of the invention. By way of example, a disclosure of from about 1.5 to about 2.0 includes the disclosure of intermediate ranges, for example, of: from about 1.5 to about 1.6; from about 1.5 to about 1.7; from about 1.7 to about 1.8; etc.

In one aspect, the present invention provides a nonwoven fabric comprising a first nonwoven layer comprising a first plurality of fine fibers, in which the first plurality of fine fibers comprise a first polymeric material including (i) a first polymer component and (ii) optionally one or more first additives. The first polymer component comprises a first recycled-polypropylene (rPP) comprising a visbroken spunbond grade polypropylene having a first melt flow rate (MFR) before visbreaking and second MFR after visbreaking, and wherein the second MFR is larger than the first MFR. In accordance with certain embodiments of the invention, the rPP utilized in any of the nonwoven layers of the nonwoven fabric (e.g., a first meltblown layer or any other layer that may be present in the nonwoven fabric) may be sourced from waste material or scraps, such as spunbond production waste. For example, a polypropylene spunbond line may produce production waste (e.g., trimmed material, low quality web formation, etc.) that may be utilized as the polypropylene source for forming, for example, meltblown layer(s) and/or melt fibrillation layer(s) of the fine fibers for the nonwoven fabric. In this regard, the polypropylene production waste from previous, for example, melt-spinning operations may be recycled (e.g., melting the production waste to form a rPP melt and forming fine fibers fibers therefrom). In accordance with certain embodiments of the invention, for example, the polypropylene production waste comprises spunbond grade polypropylene having a relatively low melt flow rate that may not be viable for the production of fine fibers, such as meltblown fibers and/or melt fibrillation fibers. In this regard, the rPP melt formed from the production waste (e.g., spunbond grade polypropylene) may also include one or more additives, such as viscosity reducing agents, in which the resulting rPP melt is larger than that of the spunbond grade polypropylene used as the source of polypropylene. By increasing the melt flow rate, for example, via viscosity reduction, the rPP may be meltblown to form a meltblown layer comprising fine fibers of rPP and/or subjected to melt fibrillation to provide a melt fibrillation layer comprising fine fibers of rPP.

In accordance with certain embodiments of the invention, the second MFR may be from about 50 to about 1500 g/10 min as determined by ASTM D1238 (230 C.°/2.16 kg), such as at least about any of the following: 50, 60, 80, 100, 120, 140, 150, 180, 200, 250, 300, 350, and 400 g/10 min as determined by ASTM D1238 (230 C.°/2.16 kg), and/or at most about any of the following: 1500, 1200, 1000, 800, 750, 700, 650, 600, 550, 500, 450, and 400 g/10 min as determined by ASTM D1238 (230 C.°/2.16 kg). Additionally or alternatively, the first MFR may be from about 20 to about 70 g/10 min as determined by ASTM D1238 (230 C.°/2.16 kg), such as at least about any of the following: 20, 25, 30, 35, and 40 g/10 min as determined by ASTM D1238 (230 C.°/2.16 kg), and/or at most about any of the following: 60, 55, 50, 45, and 40 g/10 min as determined by ASTM D1238 (230 C.°/2.16 kg). Additionally or alternatively, an MFR-ratio between the second MFR and the first MFR (i.e., second MFR:first MFR) may comprise from 5:1 to about 30:1, such as at least about any of the following: 5:1, 8:1, 10:1, 12:1, and 15:1, and/or at most about any of the following: 30:1, 28:1, 25:1, 22:1, 20:1, 18:1, and 15:1. Additionally or alternatively, the rPP, in accordance with certain embodiments of the invention, may have a first molecular weight distribution prior to visbreaking and a second molecular weight distribution after visbreaking, in which the second molecular weight distribution is not greater than 3.5 as determined in accordance with ASTM D-6474-12, such as at most about any of the following: 3.5, 3.2, 3, 2.8, 2.5, 2.2, and 2, and/or at least about any of the following: 1, 1.2, 1.4, 1.6, 1.8, 2.

In accordance with certain embodiments of the invention, the first polymer component comprises from 10% to 100% by weight of the first rPP, such as at least about any of the following: 10, 15, 20, 25, 30, 35, 40, 45, 50, 55, and 60% by weight of the first rPP, and/or at most about any of the following: 100, 99, 98, 96, 95, 94, 92, 90, 85, 80, 75, 70, 65, and 60% by weight of the first rPP. In accordance with certain embodiments of the invention, the rPP may be blended with virgin polypropylene, virgin elastomeric polymers, etc. (e.g., the balance of the first polymer component may be virgin polypropylene and/or virgin elastomeric polymers). Examples of virgin elastomeric polymers, for example, may include Vistamaxx™ polymers (e.g., Vistamaxx™ 6202), which are polypropylene-based elastomers that comprises a copolymer of propylene and ethylene. These propylene-based elastomers, for example, comprise isotactic polypropylene microcrystalline regions and random amorphous regions (e.g., ethylene). Such olefinic copolymers may comprise hard blocks and soft blocks, where the hard blocks are primarily propylene and the soft blocks are primarily ethylene. In this regard, the hard blocks (e.g., propylene) may comprise 10-90% by weight of the copolymer while the soft blocks may comprise from 90-10% by weight of the copolymer. In this regard, these copolymers include a random ethylene distribution throughout the copolymer. Vistamaxx™ (e.g., Vistamaxx™ 6202) copolymers are commercially available from ExxonMobil. Vistamaxx™ 6202 has a density of 0.862 g/cc, a MI (190° C./2.16 kg) of 9.1, a MFR (230° C./2.16 kg load) of 20, and an ethylene content of 15% by weight. An additional example includes an olefin diblock copolymer comprising an EP-iPP diblock polymer such as Intune™, which is a polypropylene-based block copolymer including ethylene monomers. By way of further examples, the polypropylene-based elastomer may comprise an EP-iPP diblock polymer that has an ethylene content from 43 to 48% by weight, or from 43.5 to 47% by weight, or from 44 to 47% by weight, based on the weight of the diblock copolymer. In an example embodiment, the EP-iPP diblock polymer may have a propylene content from 57 to 52% by weight, or from 56.5 to 53% by weight, or from 56 to 53% by weight, based on the weight of the EP-iPP diblock polymer.

In accordance with certain embodiments of the invention, the rPP melt and the resulting meltblown layer(s) and/or melt fibrillation layer(s) comprising rPP is devoid of an elastomeric polymer. In this regard, for example, the rPP and the resulting layer(s) including the fine fibers comprising rPP may be formed entirely from polypropylene (e.g., rPP, virgin polypropylene, or blends thereof).

In accordance with certain embodiments of the invention, the first polymeric material may have an aggregate MFR (e.g., total MFR based on rPP, additives, and any other polymers included in the first polymeric material) may be from about 50 to about 1500 g/10 min as determined by ASTM D1238 (230 C.°/2.16 kg), such as at least about any of the following: 50, 60, 80, 100, 120, 140, 150, 180, 200, 250, 300, 350, and 400 g/10 min as determined by ASTM D1238 (230 C.°/2.16 kg), and/or at most about any of the following: 1500, 1200, 1000, 800, 750, 700, 650, 600, 550, 500, 450, and 400 g/10 min as determined by ASTM D1238 (230 C.°/2.16 kg).

In accordance with certain embodiments of the invention, the first plurality of fine fibers may have an average diameter from 0.2 to 12 microns, such as at least about any of the following: 0.2, 0.4, 0.5, 0.6, 0.8, 1, 1.2, 1.4, 1.5, 1.6, 1.8, 2, 2.2, 2.5, 2.8, and 3 microns, and/or at most about any of the following: 12, 11, 10, 9, 8, 7, 6, 5, 4, and 3 microns.

In accordance with certain embodiments of the invention, the first nonwoven layer may comprise from a 50% to 100% by weight of the first plurality of fine fibers, such as at least about any of the following: 50, 55, 60, 65, and 75% by weight of the first plurality of fine fibers, and/or at most about any of the following: 100, 99, 98, 96, 95, 94, 92, 90, 85, 80, and 75% by weight of the first plurality of fine fibers. The fine fibers, in accordance with certain embodiments of the invention, may comprise meltblown fibers, melt fibrillated (e.g., melt-fibrillated fibers), or both. In accordance with certain embodiments of the invention, the first plurality of fine fibers comprises from 10% to 100% by weight of meltblown fibers, such as at least about any of the following: 10, 20, 30, 40, 50, and 60% by weight of meltblown fibers, and/or at most about any of the following: 100, 99, 98, 96, 95, 94, 92, 90, 85, 80, 75, 70, 65, and 60% by weight of meltblown fibers. Additionally or alternatively, the first plurality of fine fibers comprises from 10% to 100% by weight of melt-fibrillated fibers, such as at least about any of the following: 10, 20, 30, 40, 50, and 60% by weight of melt-fibrillated fibers, and/or at most about any of the following: 100, 99, 98, 96, 95, 94, 92, 90, 85, 80, 75, 70, 65, and 60% by weight of melt-fibrillated fibers.

In accordance with certain embodiments of the invention, the nonwoven fabric may comprise from about 1 to about 10 layers of the fine fibers comprising rPP as described and disclosed herein, such as at least about any of the following: 1, 2, 3, 4, and 5 layers, and/or at most about any of the following: 10, 9, 8, 7, 6, and 5 layers. In this regard, the nonwoven fabric may comprise a stand-alone meltblown fabric or melt fibrillated fabric (e.g., all fibers of the nonwoven fabric are fine fibers containing rPP as described and disclosed herein). In such embodiments, for example, the nonwoven fabric may be devoid of staple fibers, cellulosic fibers, and/or spunbond fibers.

As noted above, the first polymeric material may include one or more first additives, in which the one or more first additives may comprise a first viscosity reducing agent. The first viscosity reducing agent, for example, may comprises a first sterically hindered hydroxylamine ester, which may be a peroxide free additive, a peroxide, or combinations thereof. Such a sterically hindered hydroxylamine ester additive is commercially available as Irgatec® CR76 (i.e., a sterically hindered hydroxylamine ester in a polymer matrix from BASF). This additive, for example, reduces the viscosity of the melt, thereby controlling the degradation of the molecular weight of the polypropylene during spinning. Additionally, it narrows the molecular weight distribution, bringing the advantage of better spinability and viable formation of fine fibers from rPP. In accordance with certain embodiments of the invention, the first polymeric material may comprise from about 0.1 to about 10% by weight of the first viscosity reducing agent, such as at least about any of the following: 0.1, 0.5. 1, 2, 3, 4, and 5% by weight of the first viscosity reducing agent, and/or at most about any of the following: 10, 9, 8, 7, 6, and 5% by weight of the first viscosity reducing agent.

In accordance with certain embodiments of the invention, the first nonwoven layer and/or the nonwoven fabric may have a first basis weight from about 1 to about 150 gsm, such as at least about any of the following: 1, 3, 5, 10, 15, 25, 50, and 75 gsm, and/or at most about any of the following: 150, 125, 100, and 75 gsm.

The nonwoven fabric, in accordance with certain embodiments of the invention, may be thermally consolidated and have a bonding pattern comprising a plurality of discrete bond sites, and wherein the plurality of discrete bond sites define a first bonded area from 3 to about 30%, such as at least about any of the following: 3, 4, 5, 8, 10, 12, and 15%, and/or at most about any of the following: 30, 28, 25, 22, 20, 18, and 15%.

In accordance with certain embodiments of the invention, fine fiber containing nonwoven layer(s) may exhibit identical or superior physical properties compared to identical meltblown layers formed entirely from virgin polypropylene. For example, the first nonwoven layer may have a hydrohead-to-basis weight ratio (mbar:gsm) from about 2:1 to about 3.5:1, such as at least about any of the following: 2:1, 2.2:1, 2.5:1, 2.6:1, and 2.8:1, and/or at most about any of the following: 3.5:1, 3.2:1, 3:1, and 2.8:1. Additionally or alternatively, the first nonwoven layer has an air permeability-to-basis weight ratio (cfm:gsm) from about 1.5:1 to about 3.5:1, such as at least about any of the following: 1.5:1, 1.8:1, 2:1, 2.2:1, 2.5:1, 2.6:1, and 2.8:1, and/or at most about any of the following: 3.5:1, 3.2:1, 3:1, and 2.8:1. The air permeability may be determined according the ASTMD737-96 and hydrohead may be determined according to standard test method IST 80.8. Additionally or alternatively, the first nonwoven layer has an LSTST from about 10 to about 20 seconds, such as at least about any of the following: 10, 12, 14, and 15 seconds and/or at most about any of the following: 20, 18, 16, and 15 seconds. The LSTST method is based upon WSP 70.3 (05), but utilizing a 32 dyne n/m surface tension fluid (5 ml volume). Additionally or alternatively, the first nonwoven layer has an LSTST-to-basis weight ratio (s:gsm) from about 0.75:1 to about 1.8:1, such as at least about any of the following: 0.75:1, 0.8:1, 0.9:1, 1:1, and 1.2:1 and/or at most about any of the following: 1.8:1, 1.7:1, 1.6:1, 1.5:1, 1.4:1, 1.3:1, and 1.2:1.

In accordance with certain embodiments of the invention, the first nonwoven layer has a machine direction (MD) tensile strength at break (N/5 cm)-to-basis weight ratio (N/5 cm/gsm) from about 1.5:1 to about 3:1, such as at least about any of the following: 1.5:1, 1.8:1, 2:1, 2.2:1, and 2.5:1, and/or at most about any of the following: 3:1, 2.8:1, 2.6:1, and 2.5:1. The test method for tensile strength and elongation is WSP 110.4. Additionally or alternatively, the first nonwoven layer has a MD elongation at break from 20 to 60%, such as at least about any of the following 20, 25, 30, 35, and 40%, and/or at most about any of the following: 60, 58, 56, 55, 54, 52, 50, 48, 45, 42, and 40%. The test method for tensile strength and elongation is WSP 110.4. Additionally or alternatively, the first nonwoven layer has a cross-direction (CD) tensile strength at break (N/5 cm)-to-basis weight ratio (N/5 cm/gsm) from about from about 0.75:1 to about 1.5:1, such as at least about any of the following: 0.75:1, 0.8:1, 0.9:1, 1:1, and 1.2:1 and/or at most about any of the following: 1.5:1, 1.4:1, 1.3:1, and 1.2:1. The test method for tensile strength and elongation is WSP 110.4. Additionally or alternatively, the first nonwoven layer has a CD elongation at break from 20 to 60%, such as at least about any of the following 20, 25, 30, 35, and 40%, and/or at most about any of the following: 60, 58, 56, 55, 54, 52, 50, 48, 45, 42, and 40%. The test method for tensile strength and elongation is WSP 110.4.

In accordance with certain embodiments of the invention, the first nonwoven layer has a mean flow pore diameter from 8 to 20 microns as determined according to ASTM F 316-03, such as at least about any of the following: 8, 10, 12, and 15 microns, and/or at most about any of the following: 20, 18, and 15 microns.

In accordance with certain embodiments of the invention, the nonwoven fabric may further comprise a second nonwoven layer (e.g., a support layer for the first nonwoven layer containing fine fibers comprising rPP) bonded to the first nonwoven layer (e.g., a first meltblown layer including rPP). The second nonwoven layer may comprise a spunbond layer, a meltblown layer, a bonded carded web of staple fibers, a mechanically consolidated layer, or any combination thereof. The second nonwoven layer may comprise a synthetic polymer, such as a polyolefin, a polyester, a polyamide, or any combination thereof. For example, the synthetic polymer may comprise a virgin polypropylene, a recycled polypropylene, a virgin polyethylene, a recycled polyethylene, a virgin polyester, a recycled polyester, or any combination thereof. Additionally or alternatively, the second nonwoven layer may comprise cellulosic fibers, such as natural cellulosic fibers, synthetic cellulosic fibers, or any combination thereof.

The second nonwoven layer may comprise from 50% to 100% by weight of a virgin synthetic polymer, such as virgin polypropylene, such as at least about any of the following: 50, 55, 60, 65, 70, 75, 78, 80, 82, 85, 88, and 90% by weight of the virgin synthetic polymer, and/or at most about any of the following: 100, 99, 98, 96, 95, 94, 92, and 90% by weight of the virgin synthetic polymer. Additionally or alternatively, the second nonwoven layer may comprise from 50% to 100% by weight of a recycled synthetic polymer, such as recycled polypropylene, such as at least about any of the following: 50, 55, 60, 65, 70, 75, 78, 80, 82, 85, 88, and 90% by weight of the recycled synthetic polymer, and/or at most about any of the following: 100, 99, 98, 96, 95, 94, 92, and 90% by weight of the recycled synthetic polymer.

In accordance with certain embodiments of the invention, the second nonwoven layer may comprise one or more spunbond nonwoven layers, such as at least about any of the following: 1, 2, 3, 4, and 5 spunbond nonwoven layers, and/or at most about any of the following: 10, 9, 8, 7, 6, and 5 spunbond nonwoven layers. Additionally or alternatively, the second nonwoven layer may comprises an SMS structure or a spunbond-cellulosic-spunbond structure, in which the spunbond-cellulosic-spunbond structure comprises a mechanically consolidated material, such as a hydroentangled nonwoven material, a needle-punched nonwoven material, or air-entangled nonwoven material. The second nonwoven layer may define a first outermost layer of the nonwoven fabric.

In accordance with certain embodiments of the invention, the second nonwoven layer may have a first basis weight from about 1 to about 150 gsm, such as at least about any of the following: 1, 3, 5, 10, 15, 25, 50, and 75 gsm, and/or at most about any of the following: 150, 125, 100, and 75 gsm. In this regard, for example, the nonwoven fabric may have a basis weight (combined from the first nonwoven layer and the second nonwoven layer) from about 2 to about 300 gsm, such as at least about any of the following: 2, 6, 10, 20, 30, 50, 100, 125, and 150 gsm, and/or at most about any of the following: 300, 275, 250, 225, 200, 175, and 150 gsm.

In accordance with certain embodiments of the invention, the nonwoven fabric may comprise a third nonwoven layer. The third nonwoven layer may comprise a spunbond layer, a meltblown layer, a bonded carded web of staple fibers, a mechanically consolidated layer, or any combination thereof. In this regard, the third nonwoven layer may be bonded to the first nonwoven layer, wherein the third nonwoven layer is a second outmost nonwoven layer of the nonwoven fabric. In this regard, the first nonwoven layer may be directly or indirectly sandwiched or located between the second nonwoven layer and the third nonwoven layer. The third nonwoven layer, for example, may comprise a spunbond layer, a meltblown layer, a bonded carded web of staple fibers, a mechanically consolidated layer, or any combination thereof. Additionally or alternatively, the third nonwoven layer may comprise a synthetic polymer, such as a polyolefin, a polyester, a polyamide, or any combination thereof. Additionally or alternatively, the synthetic polymer comprises a virgin polypropylene, a recycled polypropylene, a virgin polyethylene, a recycled polyethylene, a virgin polyester, a recycled polyester, or any combination thereof.

In accordance with certain embodiments of the invention, the third nonwoven layer comprises from 50% to 100% by weight of a virgin synthetic polymer, such as virgin polypropylene, such as at least about any of the following: 50, 55, 60, 65, 70, 75, 78, 80, 82, 85, 88, and 90% by weight of the virgin synthetic polymer, and/or at most about any of the following: 100, 99, 98, 96, 95, 94, 92, and 90% by weight of the virgin synthetic polymer. Additionally or alternatively, the third nonwoven layer comprises from 50% to 100% by weight of a recycled synthetic polymer, such as recycled polypropylene, such as at least about any of the following: 50, 55, 60, 65, 70, 75, 78, 80, 82, 85, 88, and 90% by weight of the recycled synthetic polymer, and/or at most about any of the following: 100, 99, 98, 96, 95, 94, 92, and 90% by weight of the recycled synthetic polymer.

In accordance with certain embodiments of the invention, the third nonwoven layer comprises one or more spunbond nonwoven layers, such as at least about any of the following: 1, 2, 3, 4, and 5 spunbond nonwoven layers, and/or at most about any of the following: 10, 9, 8, 7, 6, and 5 spunbond nonwoven layers.

In accordance with certain embodiments of the invention, the first outermost nonwoven layer, the second outermost nonwoven layer, or both comprise a spunbond layer. For example, the first outermost nonwoven layer, the second outermost nonwoven layer, or both comprises one or more spunbond nonwoven layers, such as at least about any of the following: 1, 2, 3, 4, and 5 spunbond nonwoven layers, and/or at most about any of the following: 10, 9, 8, 7, 6, and 5 spunbond nonwoven layers.

In accordance with certain embodiments of the invention, the nonwoven fabric may comprise a spunbond-fine fiber nonwoven layer-spunbond fabric that may comprise one of the following structures:
(Structure 1) S1ₐ-M_{b}-S2_{c};
(Structure 2) S1ₐ-N_{d}-S2_{c}; and
(Structure 3) S1ₐ-M_{b}-N_{d}-S2_{c};
wherein
‘M’ comprises a meltblown layer or layer;
‘N’ comprises a melt fibrillated layer (e.g., a melt-film fibrillated layer);
‘S1’ comprises a first spunbond layer;
‘S2’ comprises a second spunbond layer;
‘a’ represents the number of layers and is independently selected from 1, 2, 3, 4, and 5;
‘b’ represents the number of layers is independently selected from 1, 2, 3, 4, 5, 6, 7, and 8;
‘c’ represents the number of layers is independently selected from 1, 2, 3, 4, and 5; and
‘d’ represents the number of layers is independently selected from 1, 2, 3, 4, and 5.

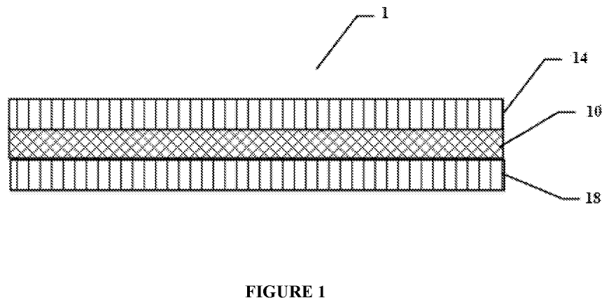

FIG. 1 illustrates a nonwoven fabric 1 including a first nonwoven layer 10 comprising a meltblown layer or melt fibrillated layer containing fine fibers located between a second nonwoven layer 14 (e.g., a spunbond nonwoven layer) and a third nonwoven layer 18 (e.g., a third spunbond nonwoven layer), in which the first nonwoven layer comprises fine fibers comprising rPP as described and disclosed herein.

In another aspect, certain embodiments according to the invention provide a method of making a nonwoven fabric, such as those described and disclosed herein, comprising the following: (a) forming a first polymeric melt comprising (i) a first polymer component, wherein the first polymer component comprises a first spunbond grade recycled-polypropylene (rPP) having a first melt flow rate (MFR); and (ii) one or more first additives, wherein the one or more first additives comprises a first viscosity reducing agent; (b) forming a first vis-broken polymeric melt comprising a step of subjecting the first spunbond grade rPP to a visbreaking operation via extruding the first polymer melt at an elevated temperature, the first spunbond grade rPP has a second MFR after the visbreaking operation, and wherein the second MFR is larger than the first MFR; (c) producing a first nonwoven layer comprising a first plurality of fine fibers, such as via a meltblowing operation and/or a melt-film fibrillation operation; and (d) consolidating the plurality of plurality of fine fibers to form the nonwoven fabric, such as those described and disclosed herein.

In accordance with certain embodiments of the invention, the elevated temperature comprises from 250° C. to 300° C., such as at least about any of the following: 250, 260, 270, and 275° C., and/or at most about any of the following: 300, 290, 280, and 275° C. Additionally or alternatively, the step of subjecting the first spunbond grade rPP to the visbreaking operation comprises a residence time from about 8 to about 20 minutes, such as at least about any of the following: 8, 10, 12, and 15 minutes, and/or at most about any of the following: 20, 18, and 15 minutes.

In accordance with certain embodiments of the invention, the first viscosity reducing agent may comprises a first sterically hindered hydroxylamine ester, a peroxide, or combinations thereof as noted above. In accordance with certain embodiments of the invention, the first viscosity reducing agent is devoid of a peroxide. Additionally or alternatively, the first polymeric melt comprises from about 0.1 to about 10% by weight of the first viscosity reducing agent, such as at least about any of the following: 0.1, 0.5, 0.8, 1, 1.2, 1.5, 1.8, 2, 3, 4, and 5% by weight of the first viscosity reducing agent, and/or at most about any of the following: 10, 9, 8, 7, 6, and 5% by weight of the first viscosity reducing agent.

In accordance with certain embodiments of the invention, the method may further comprise a step of forming or providing a second nonwoven layer, wherein the second nonwoven layer comprises a spunbond layer, a meltblown layer, a bonded carded web of staple fibers, a mechanically consolidated layer, or any combination thereof, and bonding the first nonwoven layer and the second nonwoven layer together to form a multilayer nonwoven fabric. For example, the method may comprise depositing the first plurality of fine fibers directly onto the second nonwoven layer.

In accordance with certain embodiments of the invention, the method may further comprise forming or providing a third nonwoven layer, wherein the third nonwoven layer comprises a spunbond layer, a meltblown layer, a bonded carded web of staple fibers, a mechanically consolidated layer, or any combination thereof, and bonding the first nonwoven layer and the third nonwoven layer together to form a multilayer nonwoven fabric. In this regard, the first nonwoven layer is directly or indirectly located between the second nonwoven layer and the third nonwoven layer as noted above.

In yet another aspect, certain embodiments of the invention provide an article comprising a personal hygiene article comprising a nonwoven fabric such as those described and disclosed herein. The personal hygiene article may comprises a diaper or pull-up. The personal hygiene article may include a barrier leg cuff (BLC), in which the BLC is formed at least in part by the nonwoven fabric, such as those described and disclosed herein

### EXAMPLES

The present disclosure is further illustrated by the following examples, which in no way should be construed as being limiting. That is, the specific features described in the following examples are merely illustrative and not limiting.

### 1. Example Set 1: Stand-Alone Meltblown Fabrics

As shown in Tables 1-2, five different sample meltblown fabrics were produced, in which the samples made with resins labeled ‘A’ and ‘B’ reference virgin polypropylene resins, while the samples made with resins labeled ‘rPP’ reference recycled-polypropylene used to form these meltblown fabrics.

TABLE 1

| | | | | Air | | | | | | |
|---|---|---|---|---|---|---|---|---|---|---|
| | | Op. | Basis | Perm @ | | Resistance | Penetration | | | Bubble |
| | vis-break. | Temp | Weight | 125 PA | Hydrohead | @ 32 lpm | @ 32 lpm | FD | MFPD | Point |
| Resin | Additive [%] | [C.] | [gsm] | [cfm] | [mbar] | [mm H2O] | [%] | [micron] | [micron] | [micron] |
| A (800 MFR) | 0 | 274 | 28.2 | 58.8 | 61.4 | 2.98 | 65.54 | 2.36 | 13.02 | 21.89 |
| B (35 MFR) | 1% | 274 | 26.6 | 59.3 | 69.3 | 2.72 | 68.37 | 2.49 | 14.05 | 23.73 |
| B (35 MFR) | 1% | 285 | 26.2 | 33.2 | 74.6 | 4.04 | 54.25 | 1.98 | 9.50 | 16.38 |
| rPP (60 MFR) | 1% | 274 | 26.4 | 48.7 | 66.0 | 2.72 | 68.06 | 2.32 | 12.29 | 23.55 |
| rPP (40 MFR) | 1% | 274 | 26.8 | 37.6 | 74.4 | 3.47 | 59.56 | 1.97 | 11.37 | 18.93 |

TABLE 2

| | | | | Air Perm | |
|---|---|---|---|---|---|
| | vis-break. | Op. | Basis | @ 125 PA | Hydrohead |
| | Additive | Temp | Weight | [cfm]/BW | [mbar]/BW |
| Resin | [%] | [C.] | [gsm] | [gsm] | [gsm] |
| A (800MFR) | 0 | 274 | 28.2 | 2.1 | 2.2 |
| B (35MFR) | 1% | 274 | 26.6 | 2.6 | 2.6 |
| B (35MFR) | 1% | 285 | 26.2 | 1.3 | 2.8 |
| rPP (60MFR) | 1% | 274 | 26.4 | 1.8 | 2.5 |
| rPP (40MFR) | 1% | 274 | 26.8 | 1.4 | 2.8 |

### 1. Example Set 2: Spunbond-Meltblown-Spunbond (SMS) Fabrics

As shown in Table 3, three different sample SMS fabrics were produced, in which the sample identified as ‘NO6076’ is a control sample that includes no rPP in the meltblown layer of the SMS fabric. The sample identified as ‘NO6080’ includes 20% rPP blended with the virgin polymer for the meltblown layer of the SMS fabric. The sample identified as ‘NO6087’ is another control being devoid of rPP.

TABLE 3

| | | N06076 | | N06080 | | N06087 |
|---|---|---|---|---|---|---|
| | N | (no rPP) | N | (with 20% rPP) | N | (no rPP) |
| Basis Weight [gsm] | 30 | 13.0 | 17 | 13.2 | 30 | 13.0 |
| HH @ 20 mbar/min | 30 | 18.3 | 17 | 17.4 | 30 | 16.8 |
| [mbar] | | | | | | |
| Air Perm [m3/m2/min] | 30 | 46.8 | 17 | 40.6 | 30 | 51.1 |
| LSTST [sec] | 30 | 11.9 | 17 | 15.5 | 30 | 11.2 |
| MD Stiffness | 8 | 3.5 | 5 | 3.7 | 2 | 4.0 |
| CD Stiffness | 9 | 2.4 | 5 | 2.3 | 2 | 3.3 |
| Opacity | 30 | 28.5 | 17 | 30.5 | 30 | 27.5 |
| L | 30 | 88.00 | 17 | 88.03 | 30 | 88.29 |
| a | 30 | −11.08 | 17 | −10.98 | 30 | −10.71 |
| b | 30 | −1.64 | 17 | −1.62 | 30 | −1.42 |
| dE | 30 | 2.28 | 17 | 2.21 | 30 | 1.98 |
| Mortindale embossed | 30 | 1.9 | 17 | 1.9 | 30 | 2.0 |
| Mortindale non- | 30 | 2.0 | 17 | 1.8 | 30 | 2.0 |
| emboseed | | | | | | |
| MD T [N/5 cm] | 31 | 29.4 | 17 | 29.7 | 30 | 29.4 |
| MD T [N/cm] | 31 | 5.9 | 17 | 5.9 | 30 | 5.9 |
| MD E [%] | 31 | 52.6 | 17 | 48.6 | 30 | 53.9 |
| CD T [N/5 cm] | 30 | 14.1 | 17 | 13.5 | 30 | 13.3 |
| CD T [N/cm] | 30 | 2.8 | 17 | 2.7 | 30 | 2.7 |
| CD E [%] | 30 | 52.2 | 17 | 48.5 | 30 | 48.4 |
| Modulus MD @ 2% | 8 | 44.5 | 5 | 53.8 | 4 | 50.6 |
| thickness [mm] | 8 | 0.135 | 5 | 0.130 | 5 | 0.120 |

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. A nonwoven fabric, comprising: a first nonwoven layer comprising a first plurality of fine fibers, the first plurality of fine fibers comprises a first polymeric material including (i) a first polymer component and (ii) optionally one or more first additives, wherein the first polymer component comprises a first recycled-polypropylene (rPP) comprising a visbroken spunbond grade polypropylene having a first melt flow rate (MFR) before visbreaking and second MFR after visbreaking, and wherein the second MFR is larger than the first MFR.

2. The nonwoven fabric of claim 1, wherein the second MFR is from about 50 to about 800 g/10 min as determined by ASTM D1238 (230 C.°/2.16 kg).

3. The nonwoven fabric of claim 2, wherein the first MFR is from about 20 to about 60 g/10 min as determined by ASTM D1238 (230 C.°/2.16 kg).

4. The nonwoven fabric of claim 1, wherein the rPP has a first molecular weight distribution prior to visbreaking and a second molecular weight distribution after visbreaking, wherein the second molecular weight distribution is not greater than 3.5.

5. The nonwoven fabric of claim 1, wherein the first polymer component comprises from 10% to 100% by weight of the first rPP.

6. The nonwoven fabric of claim 1, wherein the first plurality of fine fibers has an average diameter from 0.2 to 12 microns.

7. The nonwoven fabric of claim 1, wherein the first plurality of fine fibers comprise meltblown fibers, melt-fibrillated fibers, or both.

8. The nonwoven fabric of claim 1, wherein the first polymeric material includes the one or more first additives, the one or more first additives comprising a first viscosity reducing agent.

9. The nonwoven fabric of claim 8, wherein the first viscosity reducing agent comprises a first sterically hindered hydroxylamine ester, a peroxide, or combinations thereof.

10. The nonwoven fabric claim 1, wherein the first nonwoven layer has a hydrohead-to-basis weight ratio (mbar:gsm) from about 2:1 to about 3.5:1.

11. The nonwoven fabric claim 1, wherein the first nonwoven layer has an air permeability-to-basis weight ratio (cfm:gsm) from about 1.5:1 to about 3.5:1.

12. The nonwoven fabric claim 1, wherein the first nonwoven layer has an LSTST-to-basis weight ratio (s:gsm) from about 0.75:1 to about 1.8:1.

13. The nonwoven fabric of claim 1, wherein the first nonwoven layer has (i) a machine direction (MD) tensile strength at break (N/5 cm)-to-basis weight ratio (N/5 cm/gsm) from about 1.5:1 to about 3:1, (ii) a MD elongation at break from 20 to 60%, (iii) a cross-direction (CD) tensile strength at break (N/5 cm)-to-basis weight ratio (N/5 cm/gsm) from about from about 0.75:1 to about 1.5:1, and/or (iv) a CD elongation at break from 20 to 60%.

14. The nonwoven fabric of claim 1, further comprising a second nonwoven layer bonded to the first nonwoven layer, wherein the second nonwoven layer comprises a spunbond layer, a meltblown layer, a bonded carded web of staple fibers, a mechanically consolidated layer, or any combination thereof.

15. The nonwoven fabric of claim 14, further comprising a third nonwoven layer comprising a spunbond layer, a meltblown layer, a bonded carded web of staple fibers, a mechanically consolidated layer, or any combination thereof, and wherein the third nonwoven layer is bonded to the first nonwoven layer, wherein the second nonwoven layer is a first outermost nonwoven layer of the nonwoven fabric and the third nonwoven layer is a second outmost nonwoven layer of the nonwoven fabric.

16. A method of making a nonwoven fabric, comprising:
(a) forming a first polymeric melt comprising (i) a first polymer component, wherein the first polymer component comprises a first spunbond grade recycled-polypropylene (rPP) having a first melt flow rate (MFR); and (ii) one or more first additives, wherein the one or more first additives comprises a first viscosity reducing agent;
(b) forming a first vis-broken polymeric melt comprising a step of subjecting the first spunbond grade rPP to a visbreaking operation via extruding the first polymer melt at an elevated temperature, the first spunbond grade rPP has a second MFR after the visbreaking operation, and wherein the second MFR is larger than the first MFR;
(c) producing a first nonwoven layer comprising a first plurality of fine fibers via a meltblowing operation or a melt-film fibrillation operation; and
(d) consolidating the plurality of plurality of fine fibers to form the nonwoven fabric.

17. The method of claim 16, wherein the elevated temperature comprises from 250° C. to 300° C.

18. An article, comprising: a personal hygiene article including a nonwoven fabric according to claim 1.

19. An article of claim 18, wherein the personal hygiene article comprises a diaper or pull-up.

20. The article of claim 19, wherein the personal hygiene article comprises a barrier leg cuff (BLC), and the BLC is formed at least in part by the nonwoven fabric.
